(19) United States
(12) Patent Application Publication
(10) Pub. No.: US 2011/0061440 A1
(43) Pub. Date: Mar. 17, 2011

(54) DEVICE AND METHOD FOR METERING MATERIALS INTO A CARRIER MATRIX

(76) Inventors: Klaus Breuer, Aschau (DE); Stefan Mair, Wachlehen (DE)

(21) Appl. No.: 12/672,205

(22) PCT Filed: Aug. 5, 2008
(86) PCT No.: PCT/EP08/06434

§ 371 Date: Nov. 29, 2010

(30) Foreign Application Priority Data
Aug. 8, 2007 (DE) 10 2007 038 278.4

(51) Int. Cl. G01F 1/34; G01N 33/00

(52) U.S. Cl. 73/1.05; 73/861.42

(57) ABSTRACT

A material transport and event control in systems with piezoelectrically activated droplet emission including a device for metering a material into a carrier matrix, having a carrier matrix source (0) supplying a carrier matrix, a supply vessel (1) filled with the material in preferably liquid form, a metering unit (3, 4) including a metering chamber (4), disposed downstream of the carrier matrix source and a metering head (3 disposed downstream of the supply vessel and configured preferably for piezoelectrically activated droplet emission, the carrier matrix being supplied to the metering chamber from the carrier matrix source, and the material being supplied to the metering head from the supply vessel, being emitted via the metering head into the metering chamber and consequently being metered into the carrier matrix, including a pressure control device configured to control the pressure in the supply vessel (1) and/or the pressure in the metering unit.

### BACKGROUND

The present invention relates to a device and also to a method for metering a material present in particular in liquid form into a carrier matrix, in particular into a gas flow. The metering unit used in the method or in the device for metering the material into the carrier matrix is hereby configured advantageously for piezoelectrically activated droplet emission.

In many fields of chemical analysis but also in production processes, it is often important to meter ultrasmall, thereby stable and reproducible concentrations of one or more materials into a carrier matrix, e.g. a gas flow or a gas volume. According to the state of the art, generally test gases are used for this purpose, which are produced by weighing the corresponding materials into a defined gas volume. This method is tedious in production and in particular in quality assurance since the longterm stability must be ensured. Often large volumes must also be produced for technical production reasons in comparison with the actually required quantity.

Further methods which make use of the principles of evaporation and permeation are likewise used. These methods have the disadvantage that they cannot generally be applied for material mixtures. The boundary conditions must also be controlled very exactly in order to avoid too great variations in the emitted material quantity.

For these reasons, people are moving over more and more to producing the required material flows directly by piezoelectrically activated droplet emission. Such methods are described for example in the patents US 2002139167 and WO 2005/0525721. The basic arrangement for such a unit comprises a supply vessel, in which the material to be metered is stored, and a metering head which is similar to those used in inkjet printers and from which the droplets are emitted by piezoelectrically induced contraction. The supply vessel and the metering head are connected to each other via a capillary. In the latter, the material to be metered is transported to the metering head. The metering head is generally placed in an arrangement which enables correct control of the carrier matrix flow into which the material to be metered is introduced. There is understood subsequently by the metering chamber the air chamber surrounding the metering head into which the droplets are emitted from the metering head. What volume the metering chamber occupies precisely varies according to the system arrangement. There is understood subsequently by a material to be metered an individual material (subsequently also termed single material) just like a material mixture, i.e. a mixture of various single materials.

In the above-mentioned state of the art, it is proposed to determine the quantity of material introduced gravimetrically by pre-tests. This method however involves a significant risk of error, for instance by the variation in droplet size which can occur in particular after switching off and again switching on, or by wall effects. In addition, significant weighing errors can be expected in the generally small quantities which are used. A change cannot be made rapidly from one material to be metered to another either since a material flow determination always requires to be implemented in advance. In practice it is shown that the droplets emitted from the metering device in the course of a metering, in particular if this last for a fairly long time can vary systemically in their size, whereby the material flow of material to be metered also varies. It is also often desired to produce different material flows within a relatively small time interval. This is not possible with the above-mentioned state of the art or requires at least significant time-consuming preparation.

In the above-mentioned state of the art, the droplets of material to be metered are introduced into a gas flow. Either the supplied gas flow or the surface on which the droplets impinge are thereby heated in order to ensure complete evaporation. At the beginning of a metering process, the liquid supply line to the metering head must be rinsed with the material to be metered. As a result, in comparison with the subsequent operation, a large quantity of substance to be metered is introduced into the system. This can be discharged with the carrier gas flow through the system. This procedure involves the danger that all the walls of the system are super-saturated with the material to be metered and then emit the latter slowly and in an uncontrolled manner back to the carrier gas in actual operation, which effects a systematically too high concentration of material to be metered during operation.

The devices according to the state of the art provide in addition direct use of the loaded carrier gas flow. This means that the carrier gas flow is conducted without further dilution to the point of use, in the case of US 2002139167 a calibration device, and in WO 2005/052571 a test chamber for moisture sensors. In the described applications, it must be stressed that, because of the limited frequency of the droplet emission, the limited droplet size and the generally limited carrier gas flow, the resulting concentrations are also limited. The concentration of material to be metered in the carrier matrix can also take place directly by dilution of the material to be metered, for instance by producing an ethanolic solution. However, it is often not desired that another material is added to the carrier matrix, which would likewise be detected for example during olfactory tests in addition to the material to be metered. It can therefore be entirely sensible to design the material to be metered as a mixture.

It is now the object of the present invention to improve the existing devices, in particular those devices which are based on piezoelectrically activated droplet emission (and also the corresponding methods) in such a manner that a desired material quantity can be added exactly to the carrier matrix in a simple, reproducible and controlled manner. Furthermore, it is the object of the present invention to make available suitable measures and arrangements in order to enable controlled and reproducible transport of material to be metered and to implement the controlled loading and guidance of the carrier matrix flow.

### SUMMARY

The basic concepts of the present invention are now firstly described subsequently. Then a brief description of individual embodiments of the present invention follows, followed by a detailed description of different embodiments of the invention.

The individual embodiments according to the invention or individual features according to the invention of the presented examples can hereby occur not only in a combination, as is shown in the special advantageous embodiments, but also can be configured or used within the scope of the present invention in any other combination possibilities.

An essential aspect of the solution to the object according to the invention is based on observing the physical conditions during metering, in particular on observing the different pressures or pressure events occurring in a metering device: as a result of different actions or physical conditions, different pressures occur at different parts of the arrangement. These are checked and possibly regulated or influenced according to the invention for a correct and reproducible metering process. Of particular interest according to the invention are:

the pressure which is present in the supply vessel in the gas phase over the material to be metered (p_{G}),

the pressure which prevails in the metering chamber (p_{R}),

the pressure which results from the difference in level between the liquid level in the supply vessel and the outlet point of the droplets (Δp_{L}).

In order to ensure a constant metering process, both p_{G}-p_{R} and Δp_{L} should advantageously be kept constant during the metering, Δp_{L} generally being much smaller than the difference between p_{G} and p_{R}. The individual pressures, pressure ratios and/or pressure differences can vary and can be determined via pre-tests for the respective substance to be metered, taking into account the total system.

The metering chamber cannot generally be kept completely without different pressure relative to the environment since there is conducted through it e.g. a gas flow which is subjected to material to be metered. Adjacent to the location of the droplet emission there generally follows an apparatus which serves to use the gas subjected to the material. This represents a flow resistance which must be overcome by a corresponding pre-pressure at the location of the droplet emission. This pressure can vary, for instance by variation in the pre-pressure in front of the metering chamber, or by means of the quantity of material to be metered which is introduced in particular at the start of the process and which can evaporate suddenly because of the temperature set in the metering chamber. It can also be possible that the pressure in the metering chamber is not known exactly since the following apparatus which serves for using the gas subjected to the material has an unknown air resistance. For the mentioned reasons, it is advantageous to ensure a pressure equalisation between supply vessel and metering chamber.

Between the outlet point of the droplets on the metering head and the supply vessel, a constant pressure difference which does not vary short-term must be overcome in order to ensure a suitable flow of the material to be metered. This results from the flow resistance which the capillary of the substance to be metered presents and from the surface tension of the substance to be metered which must be overcome during the droplet emission at the tip of the metering head. In fact, this takes place mainly because of the pressure generation by the piezo unit, however tests show that a specific increase in the pressure difference can promote the droplet emission. This must be tested for each material to be metered. In metering processes which last for a fairly long time, the liquid level in the supply vessel drops. As a result, the described pressure difference is reduced. This must be compensated for in order to maintain constant metering.

It is required for starting and ending the metering process to fill or to empty the capillary. There thereby applies: p_{G}>>p_{R} (filling of the capillary) and p_{G}<<p_{R} (emptying of the capillary). Since the pressure differences occurring between p_{G} and p_{V} much greater than Δp_{L}, this value can be neglected during filling and emptying processes.

At the beginning of a metering process, the line or capillary between supply vessel and metering head must be filled with the material to be metered. For this purpose, it is necessary to apply a sufficiently high pre-pressure at the supply vessel. As tests show, a single rinsing process often does not suffice to wet the capillary inner wall homogeneously with the substance to be metered. However this is absolutely necessary in order to achieve a correct metering process. It is hence sensible to repeat the rinsing process, it proving to be sensible to empty the capillary respectively before a new rinsing process is implemented. In order to empty the capillary, a sufficiently great low pressure must be applied to the supply vessel, said low pressure conveying the liquid back into the supply vessel.

A similar process should be effected after completion of one metering. Firstly, the material to be metered must be removed from the capillary, for which purpose a sufficiently great low pressure must be applied again in the supply vessel. It is possibly sensible to exchange the supply vessel for an empty vessel in order to remove all the remaining residues of the material to be metered by again applying low pressure, now to the empty vessel, from the capillary. Rinsing the capillary with a suitable rinsing liquid is also sensible. For this purpose, a vessel with rinsing liquid is connected instead of the supply vessel. A plurality of rinsing and emptying processes follow analogously to the above-described starting process.

For a more precise description of the present invention, firstly the measures undertaken with respect to the pressure regulation according to the invention are divided into three categories:

1) equalisation of the pressure between the supply vessel and the metering chamber,

2) equalisation of the pressure which is produced from the difference in level between the liquid level in the supply vessel and the outlet point of the droplets (during exit into the metering chamber) and

3) production of the starting and rinsing process.

### Equalisation of the Pressure Between Supply Vessel and Metering Chamber

In this variant according to the invention, a pressure control device is provided, which enables a pressure equalisation between the supply vessel and the metering chamber. Such a pressure control device can be produced in the simplest case by an open gas transfer line between the metering chamber and the supply vessel. By means of such a transfer line, a gas exchange between the two volumes of the metering chamber and the supply vessel is made possible, which leads to very rapid pressure adaptation. However, material to be metered which has been evaporated either already in the metering chamber or is in the supply vessel in the gas phase above the material to be metered can hereby also be transported. In the individual case, this can in fact be acceptable but generally is a non-desired effect: in order to prevent such a transfer of material to be metered in the gas phase between supply vessel and metering chamber, a device can advantageously be incorporated according to the invention in the pressure equalisation line, which device in fact allows a pressure equalisation, as described, but at the same time prevents the passage of material to be metered.

A further possibility according to the invention for implementing the pressure equalisation between supply vessel and metering chamber by means of a pressure control device is presented by the use of pressure sensors for the pressures in the metering chamber and/or in the supply vessel. With such pressure sensors, the pressures in both volumes can be determined and the pressure difference which is calculated therefrom and hence known between pressure chamber and supply vessel can be used for the purpose of correspondingly adjusting the pressure in the supply vessel (and/or also in the metering chamber). It is thereby not necessary to construct a direct gas path between metering chamber and supply vessel (as is the case in the previously described variant), which precludes transfer of material to be metered. Such a gas path can however be fitted in addition. Rather, the pressure equalisation is effected in this case, as described subsequently in more detail, by additionally fitted systems (a control unit connected to the pressure sensor and also an adjustment unit connected to the control unit).

Equalisation of the Pressure which Results from the Difference in Level Between the Liquid Level in the Supply Vessel and the Outlet Point of the Droplets

In a further embodiment of the invention, a pressure control device can be provided, with which the pressure resulting from the difference in level between the liquid level in the supply vessel and the outlet point of the droplets is adjusted. This can take place according to the invention in different ways. The simplest hereby is to adapt the relative height between the liquid level in the supply vessel and the point of the droplet emission to the material to be metered and to keep it constant if required for the entire course of the metering. This can be achieved for example via manual post-control or also via a mechanical adjustment device.

According to the viscosity, the material must overcome a specific resistance for example in a capillary system. The latter can be determined or estimated and the device correspondingly fitted, e.g. via a mechanical adjustment device in the form of a threaded rod with an adjustment nut.

In a line between the supply vessel and the metering head, a shut-off device can hereby be integrated which is suitable for shutting off the liquid flow. In addition, a pump can be installed in this line (for liquid transport), the shut-off device is then advantageously disposed between the supply vessel and the pump. This can take place in particular in the form of a control valve which is suitable for controlling the liquid flow in a suitable manner.

It is of course possible to connect a plurality of supply vessels to a metering head by means of suitable line circuits and/or valves. It is likewise possible to connect a plurality of metering heads to a supply vessel by means of suitable line circuits and/or valves. Advantageously, a separating device can be integrated furthermore in the liquid line between supply vessel and metering head, which separating device is suitable for separating particles from the material to be metered or from the liquid to be metered.

A further possibility according to the invention for producing a pressure control device configured in this manner resides in undertaking the adjustment of the suitable pre-pressure by means of a pump. Such a pump must be able to produce a constant, thereby relatively low, pre-pressure and also to maintain this. The advantage of such a solution resides in the fact that, with such a pump, processes already described above or still to be described subsequently can be implemented partially or completely. The pump is hereby integrated advantageously in the liquid line between supply vessel and metering head. Advantageously, the pump is thereby suitable for moving the liquid to be metered both in the direction of the metering head and in the direction away from the latter. As an alternative hereto, also two suitable pumps can be integrated between supply vessel and metering head. One of the pumps is then hereby suitable for moving the liquid to be metered in the direction of the metering head and the other pump is suitable for moving the liquid to be metered in the direction away from the metering head. Furthermore, an excess pressure valve can be integrated in the line between pump and metering head (or between the pump arrangement comprising two pumps and the metering head). A plurality of supply vessels can be connected in turn to one metering head by means of suitable line circuits and valves or also a plurality of metering heads can be connected to one supply vessel. A shut-off device can finally be integrated in the line between pump (or pump arrangement) and metering head, which shut-off device is suitable for preventing the liquid flow. In addition a shut-off device which is suitable for separating particles from the liquid likewise can be integrated.

A third possibility according to the invention for configuring a corresponding pressure control device in order to produce the required pre-pressure is to place the supply vessel a priori exceptionally high, which means producing with certainty too high a pre-pressure. This is then reduced again by a suitable arrangement between supply vessel and metering head so far that a suitable static pre-pressure prevails at the point of the droplet emission. This can be produced again manually, for instance by actuating a needle valve or by a suitable mechanical device (e.g. pressure control valve).

Production of a Starting and Rinsing Process with the Above-Described Variants

As already described for starting and after completion of the metering process, the capillary or the connection line between supply vessel and metering head must be possibly alternately rinsed and emptied. Such rinsing and emptying processes can be jointly implemented, as described above, by choosing a suitable pump. The possibility must thereby be provided of producing a liquid flow in both directions, both from the supply vessel to the metering head and vice versa.

The use of a piston instead of a pump (see also subsequent description) which can be actuated either manually or by suitable mechanics is hereby easier. With such a piston, both a low and a high pressure can be produced in the supply vessel. If in addition a suitable electronic control device or control unit is used for control of such a piston, the above-described processes according to the invention can likewise be implemented therewith. In order to be able to prevent the application of the high or low pressure produced in the piston at any time, a shut-off device is advantageously installed between piston and supply vessel.

### Dilution According to the Invention and Resulting Possibilities for Using the Loaded Carrier Matrix Flow

A further essential aspect of the present invention (as is described subsequently in more detail is of making available a plurality of carrier matrix supply stretches (subsequently also termed carrier matrix supply channels) which are provided respectively with measuring and/or control devices and also the subsequent combining of the carrier matrices conducted through the plurality of carrier matrix supply channels and also of conducting the combined carrier matrix flows for subsequent use.

The loaded carrier matrix is hereby diluted in one or more further steps before use thereof. If sufficiently precise systems are used for measurement and control of the carrier matrix flows involved, then the resulting concentration in the then diluted carrier matrix can be calculated with sufficient accuracy. By means of the coupling of a plurality of such dilution steps, effected advantageously according to the invention, it is possible to generate even the smallest concentrations reliably. The possibility is likewise presented of using the resulting branch flows specifically when implementing a plurality of dilution steps, e.g. for calibration processes, since the branch flows are present in a defined (known) ratio relative to each other.

### Further Advantageous Embodiment Possibilities of the Present Invention

In the case of all the previously presented devices or methods according to the invention, measurements of the resulting concentration of material to be metered in the carrier matrix can advantageously be effected: the specific addition of materials into a carrier matrix by means of a piezo-activated droplet emission can often involve a non-stationary process which varies either when desired or on the basis of inherent conditions in the system in the course of a metering. Checking such a process is possible in the most simple manner by a simultaneous measurement (online measurement) of the concentration, generated by the droplet emission, of the material to be metered in the carrier matrix. A sufficiently contemporary measurement can also possibly suffice. There can thereby be used as measuring system or as concentration measuring unit both a system which deduces the carrier matrix loaded with material for analysis of the concentration and a system which can determine the concentration without taking a sample.

Example of a system which operates without taking a sample: infrared measuring system. Example of a system which operates with taking a sample: process-FID system (FID=flame ionisation detector).

If a measuring system which removes carrier matrix for analysis is hereby chosen, then the removed quantity must be taken into account in the calculation of the material flows since it cannot be supplied generally any longer for actual use of the system. This can take place via a suitably configured control unit. The concentration monitoring can hereby be implemented continuously or at sufficiently small intervals of time.

In all previously presented variants, a discharge of unrequired, metered material can advantageously be effected from the system. In the course of a metering process, situations can occur in which the quantity of material to be metered which is introduced into the system is much higher than the quantity actually introduced in operation. In such cases, it is advantageous to remove the excess material introduced as quickly as possible from the system. For this purpose there can be integrated in the system a gas path for discharging carrier matrix which is loaded with material and not provided for the actual operation. This is also advantageous when for example the concentration of the material to be metered in the carrier matrix is intended to be changed. Concentration peaks can hereby occur which are then discharged. Such a discharge device can also be used for possibly necessary rinsing processes of the system in the course of which the concentration of material to be metered in the system is intended to be brought towards 0 (for example zero setting).

The devices provided for discharge or outflow of unrequired metered material or loaded carrier matrix are subsequently also termed more precisely reject channels.

### BRIEF DESCRIPTION OF THE DRAWINGS

Subsequently, it is now described firstly how control of the addition of material by means of the metering head is effected according to the invention in concrete terms. There are shown in this respect:

FIG. 1 a first embodiment of the invention using a transfer line.

FIG. 2 a second embodiment of the invention using pressure sensors and also a high pressure and a low pressure line.

FIG. 3 a further embodiment using pressure sensors and also using a piston or a pump.

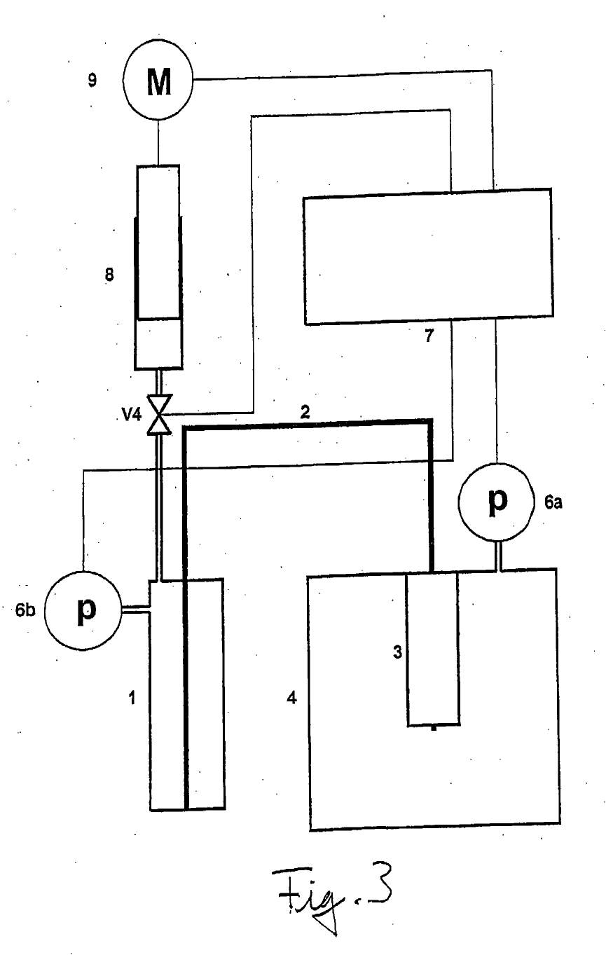

FIG. 4 a further embodiment using a pump arrangement in the liquid line between the supply vessel and the metering head.

It is described subsequently how the control and/or adjustment of the already loaded carrier matrix flow, in particular by dilution of the same, can be effected in concrete terms. There are shown in this respect:

FIG. 5 a first embodiment with two carrier matrix supply channels with respectively integrated measuring and/or control device.

FIG. 6 a further embodiment in which the device shown in FIG. 5 is supplemented by further inflow channels and outflow channels.

### DESCRIPTION OF PREFERRED EMBODIMENTS

### Embodiment 1

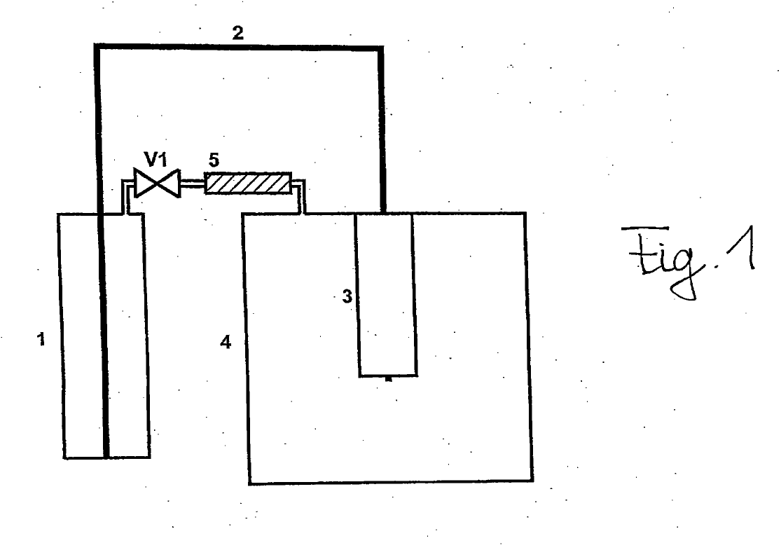

FIG. 1 shows a first embodiment of a device for metering a material into a carrier matrix according to the invention. A supply device 1 which is connected via a line, here a capillary line 2, to the metering head 3 of a metering unit is hereby provided. The metering unit has furthermore, in addition to the metering head 3, a metering chamber 4 which represents a chamber in which the metering head 3 is disposed in order to emit the material into the metering chamber 4. The metering head concerns a metering head which is configured for piezoelectrically activated droplet emission.

According to the invention, the illustrated device now has a connection Ü in the form of a transfer line between the supply vessel 1 and the metering chamber 4. This connection is basically configured as an open gas connection but can also be closed in a gas-tight manner by means of a shut-off device in the form of a valve V1. The valve V1 is hereby integrated in the transfer line Ü. In the gas path between valve V1 and metering chamber 4, a material shut-off device S is integrated furthermore in the transfer line Ü.

In the present case, the pressure equalisation between supply vessel 1 and metering chamber 4 is hence produced by a transfer line Ü. This transfer line is designed with a sufficiently large inner diameter in order to enable a sufficiently rapid pressure equalisation. At the same time, the inner diameter is hereby kept as small as possible in order to limit the material transfers between supply vessel and metering chamber, which are unavoidable with this approach according to the invention, to have as small a value as possible.

Furthermore, the possibility is offered that the gas path between supply vessel 1 and metering chamber 4 is shut off: this takes place via the valve V1. This can be for example necessary in order to implement the above-described starting process.

In the present case, the system has furthermore a material shut-off device 5 which is fitted in the gas path between supply vessel and metering chamber 4: the purpose of this device 4 is to prevent passage of material to be metered, but simultaneously to permit a pressure equalisation. There can be used as material shut-off device 5, suitable adsorbtive and/or absorbent or catalytically active substances which are introduced into the transfer line Ü. These have the effect that material to be metered is deposited or decomposed by the air flowing through the transfer line. The choice of substances is hence based of course according to the material to be metered. It must hereby be ensured that the flow resistance is sufficiently low so that a sufficiently rapid pressure equalisation can be effected. This can be achieved for example by suitable particle size of the substance.

A further possibility for preventing the passage of substance to be metered is the use of a membrane as material shut-off device 5 in the transfer line Ü. This membrane must hereby be dimensioned as a function of the pressure variations to be expected such that even the greatest possible pressure variations can be compensated for without the membrane tearing. Alternatively, it is also conceivable to use a liquid as separating device or as material shut-off device 5. It must hereby be ensured that the pressure equalisation can be effected in both directions. This can be achieved for example by configuration of a part of the transfer line in the form of a U-pipe, the U-pipe section of the transfer line Ü having a sufficiently large thickening at both ends so that blowing out the liquid is prevented. In this case, passage of material to be metered would not however be precluded. In such a case, the liquid must hence be placed in an arrangement which is suitable for allowing a gas exchange between metering chamber and the gas phase above the liquid to be metered in the supply vessel without liquid consumption.

In the illustrated embodiment (this applies likewise to the subsequently illustrated embodiments), the carrier matrix source (which can also comprise a plurality of single sources), with which a carrier matrix can be produced in the form of a gas flow and can be supplied to the metering chamber 4 for metering the material, is not shown. The material to be metered is contained in the supply vessel 1 in liquid form. In the illustrated case, the pressure control device according to the invention is hence configured via the elements Ü, V1 and 5.

Furthermore, it is possible, as an alternative to the open gas variant, that a device for pressure equalisation between metering chamber 4 and the gas phase above the liquid to be metered is provided in the form of a gas-tight transition. As already described, such a device for pressure equalisation can concern a suitably configured membrane. In turn, a shut-off device can be integrated in the transition then configured to be gas-tight, which shut-off device is suitable for preventing the pressure equalisation.

### Embodiment 2

In this case, the pressure control device according to the invention, as described subsequently in more detail, is configured by means of two pressure sensors 6a and 6b, a pressure supply line 11, a low pressure line 11a, a high pressure line 11b, two valves V2 and V3 and a control unit 7. The control unit 7 thereby serves, as a function of the pressures detected by the pressure sensors 6a and 6b, for controlling the pressure in the supply vessel 1 by means of the adjustment unit 11 (which is configured here in the form of low pressure and high pressure lines, however it can also be configured, in the subsequently also described examples, also alternatively by the elements 8, 9 and/or 10).

The first pressure sensor 6a is connected to the metering chamber 4 and detects the pressure prevailing in this chamber. The second pressure sensor 6b is connected to the supply vessel 1 and detects the pressure above the liquid level in the supply vessel. The measured pressures are conducted via lines to the control unit 7. The control unit 7 evaluates the pressure difference between the two detected pressures and accordingly controls the pressure in the supply vessel 1 by its connection to two valves V2 and V3: for this purpose, the supply vessel 1 is connected via the pressure line 11 to a low pressure line 11a and a high pressure line 11b. In the low pressure section 11a, the valve V3 is provided, in the high pressure line section 11b the valve V2. Therefore according to the detected pressure state or according to the detected pressure difference between supply vessel and metering chamber, either the valve V2 is opened and the valve V3 closed or vice versa via the control unit 7. In the first case, the supply vessel 1 is supplied with a high pressure p+, in the reverse case with a low pressure p−. The further embodiment of this metering device corresponds to the case shown in FIG. 1.

This illustrated variant has the advantage that the transfer of material to be metered by the use of pressure sensors for sensing the pressure in the metering chamber (sensor 6a) and the pressure in the metering vessel (sensor 6b) is precluded since no transfer line Ü is required. In the case of this system, the pressure in the supply vessel 1 is regulated by means of the electronic control and regulation device or control unit 7 on the basis of the pressure difference detected by the pressure sensors and hence known. This is effected in that the two shut-off devices V2 and V3 are opened or closed according to requirements. The shut-off device V2 thereby opens the supply line to the supply vessel at which high pressure prevails. The shut-off device V3 opens the supply line to the supply vessel at which low pressure prevails. High pressure can for example be produced in that a pressure line 11b is connected or in that alternatively a pump produces a sufficient pre-pressure. This pre-pressure must be sufficiently above the pressure in the metering chamber 4 in order to be able to implement the required equalisation processes. Low pressure can be for example produced in that a piston, which produces a constant low pressure by corresponding withdrawal, or a pump is connected to the low pressure line 11a. It is hereby also possible that a high pressure is constantly applied in the metering chamber 4 as a function of the construction. In this case, production of low pressure can possibly be dispensed with. The pressure difference relative to the ambient air can also be sufficient for the corresponding equalisation processes.

Regulation of the pressure in the gas phase above the liquid to be metered in the supply vessel 1 is hence effected by opening and/or closing respectively a gas line with low pressure and a gas line with high pressure. The low pressure can hereby be produced by a pump, a piston or by the available pressure difference between metering chamber and the gas phase above the liquid to be metered in the supply vessel. The same applies to the required high pressure. Furthermore, a further shut-off device which is suitable for preventing the gas flow can be integrated in the supply line 11.

### Embodiment 3

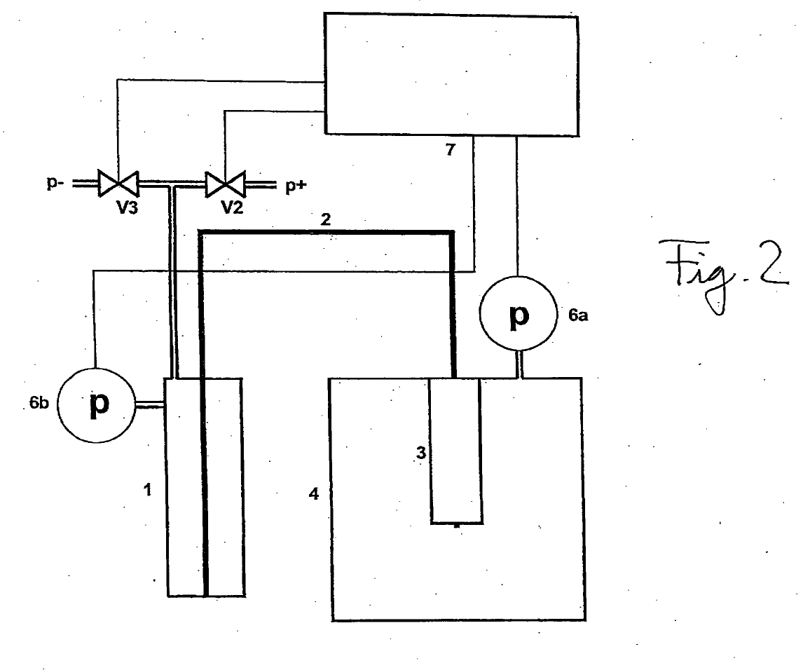

The subsequently described device is basically constructed just like the device shown in FIG. 2. Therefore only the differences are described. Instead of using two inputs 11a and 11b, respectively for low and high pressure, the production of high or low pressure in the supply vessel 1 is produced in this case by means of a piston 8k. As an alternative thereto, the high or low pressure can also be effected by means of a pump 8p which is disposed instead of the piston (not shown here). Only one gas line 12 between the piston 8k or the pump 8p and the supply vessel 1 is hereby provided. The piston or the pump is connected to a servomotor 9 which is controlled in turn via the control unit 7. Furthermore, the line 12 between piston/pump and supply vessel 1 has a shut-off device V4 in the form of a valve. This is likewise controlled via the control unit 7.

In the present case, the piston 8k produces high pressure by compression and low pressure by expansion (control by means of the motor 9). The dimensioning of the piston 8k must thereby be adapted to the size of the supply vessel. The drive of the piston can be effected, as described, via the servomotor 9, i.e. via a mechanical device, but it can also be operated manually. An electrically actuatable movement system is hence produced here, in which the pressures detected via the pressure sensors 6a and 6b are evaluated in the control unit 7, whereupon the adjustment unit 8k, 9, V4 and 12 is adjusted by means of the control unit 7 in order to produce a suitable pressure in the supply vessel 1. Hence an automated pressure control is possible with the described elements.

In order to interrupt the gas path between supply vessel 1 and piston 8k, the use of the shut-off device V4 is provided. When using the piston 8k and a suitable control and regulation device or control unit 7 with the described pressure sensors 8, all the pressure events described already in the above sections can be implemented or checked. When using a pump instead of the piston, this must be chosen such that it can produce both sufficient high pressure and sufficient low pressure. It must thereby be able to change sufficiently rapidly between the production of high pressure and the production of low pressure. It can be then connected, analogously as with the piston, to the electronic control and regulation device 7.

### Embodiment 4

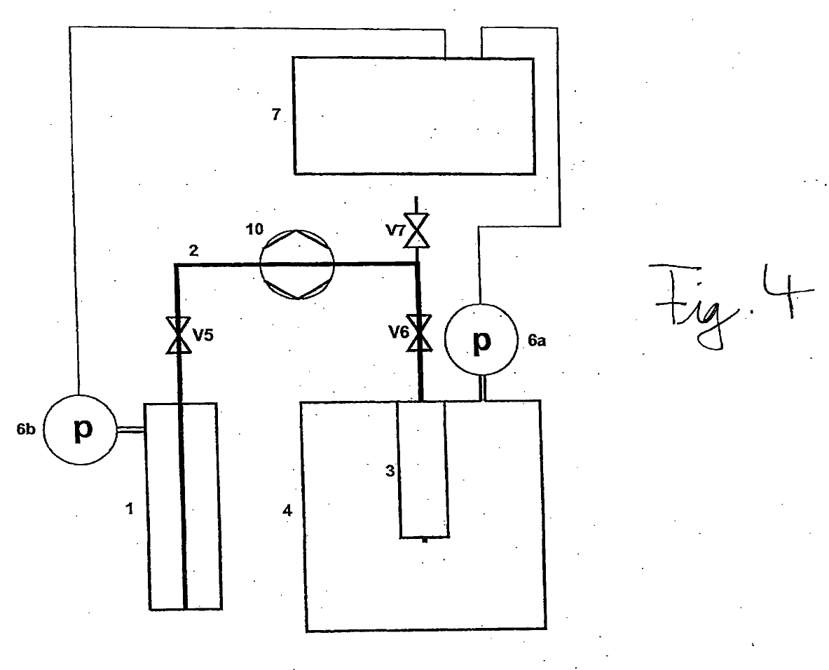

FIG. 4 shows a further embodiment of the present invention. The device shown here is basically constructed just like the device shown in FIG. 1 so that only the differences are described subsequently.

The supply vessel 1 is connected to a pressure sensor 6b; the metering chamber 4 to a further pressure sensor 6a (analogously as with the previous two examples). The pressure sensors are in turn connected to the control unit 7. However, the control unit 7 now, as described subsequently in more detail, controls a liquid pump 10 which is fitted in the liquid line 2 between supply vessel 1 and metering head 3. There is situated between liquid pump 10 and metering head 3, in the mentioned line, a shut-off device V6 in the form of a valve. A further shut-off device V5 in the form of a valve is situated in the line part between supply vessel 1 and the metering pump 10. In the region between the metering pump 10 and the shut-off valve V6, the line 2 is provided furthermore with a high pressure valve V7.

In this illustrated case, a liquid pump 10 is hence used to control the above-described pressure events. The liquid pump is hereby able to move the liquid in two directions, i.e. from the metering head to the supply vessel and in the reverse direction. Alternatively, also two liquid pumps can be used, the first liquid pump then conveying the liquid in the direction of the metering head and the second liquid pump conveying the liquid away from the metering head. Corresponding coordination of the liquid pumps must then be ensured. The liquid pump or the liquid pump arrangement (comprising both liquid pumps) must be able to maintain a slight high pressure, however possibly also to produce a sufficiently high flow towards the metering head or away from the metering head. In order to compensate for temporarily occurring pressure variations in the metering chamber 4, either the liquid pump 10 (or the liquid pump arrangement) can be correspondingly dimensioned, i.e. a sufficiently rapid switching of the liquid flow direction must be possible (the dimensioning is hereby effected of course by means of the line diameter of the line 2, the volumes of the supply vessel 1 and of the metering chamber 4 and also by means of the configuration of the metering head 3) or, as shown here in addition, the excess pressure valve V7 is integrated between the liquid pump 10 and the metering head 3 in the liquid line 2. If such an excess pressure valve V7 is used, it must be ensured that it is not triggered with the desired pressure build-up between liquid pump 10 and metering head 3. The excess pressure valve V7 can also be disposed between the supply vessel 1 and the pump 10 on the line 2.

In order to interrupt the liquid flow between supply vessel 1 and metering head 3, the shut-off devices V5 and V6 are disposed here. Furthermore, it is possible (not shown) to integrate a filter device, e.g. a frit, in the liquid line 2. Since the conduction of liquid in the metering head 3 must be effected in a very low-viscosity manner, it is correspondingly susceptible to contamination by solid particles. These can then be deposited by using such a filter device and hence do not reach the region of small line diameters in the vicinity of or in the metering head. The use of a liquid pump or liquid pump arrangement 10, as shown, also makes it possible to connect an arrangement of a plurality of supply vessels, instead of a single supply vessel 1, to a metering head 3, which can be advantageous according to the application case.

With the device shown in this example, the above-described rinsing process can also be automated, in that the material to be metered into the supply vessel 1 is pumped back until the liquid line 2 is adequately emptied. Via a suitable liquid distribution arrangement from a rinsing liquid supply vessel (not shown), rinsing liquid can thereupon be conveyed through the liquid line 2 into the metering head. By repeated pumping of the liquid line until empty, the rinsing liquid can then also be removed. For complete emptying of the liquid line 2, it can be sensible subsequently to apply a low pressure to the liquid line 2 in order to remove any last residues of rinsing liquid.

The solutions described in the above-illustrated embodiments 1 to 4 can also be used in combination or in a common system.

### Embodiment 5

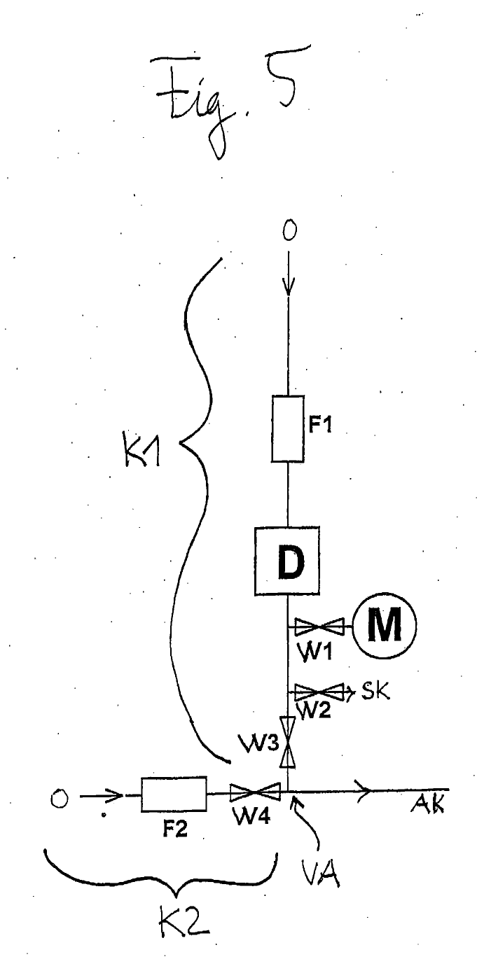

FIG. 5 shows a further embodiment of a device for metering a material into a carrier matrix. The device shown here has a carrier matrix source 0 which is constructed here as a multiple supply unit such that it supplies both carrier matrix supply channels K1 and K2, which are described subsequently in more detail, on the basis of suitable line and/or valve control. The carrier material supply channel K1 here, viewed in flow direction, has firstly a first measuring and/or control device F1. To the latter, a metering device D is connected subsequently downstream, which can be configured as described in one of the previously described embodiments 1 to 4. Downstream of the metering device D, a concentration measuring unit M is configured in the first carrier matrix supply channel K1, with which concentration measuring unit the concentration of the material in the carrier matrix loaded by means of the device D can be detected. This concentration measuring unit M is connected via a shut-off device (valve) W1 to the part of the supply channel K1 which is disposed downstream of the metering device D. Downstream of the concentration measuring unit M, a reject channel SK leads via a further valve W2 out of the channel K1 with which the excess carrier matrix which is loaded with the material and not intended for use, described more subsequently, can be discharged out of the first supply channel K1. Finally, the first supply channel K1, on the downstream side of the oufflowing reject channel SK, has a further shut-off device in the form of a valve W3.

Furthermore, the illustrated device has a second carrier matrix supply channel K2 to which carrier matrix can be supplied likewise via the carrier matrix source 0. This second supply channel K2 has firstly a second measuring and/or control device F2 likewise for measuring and/or controlling the carrier matrix flow (here the carrier matrix flow leading through the second carrier matrix supply channel K2). Downstream of the control device F2, likewise a valve W4 is disposed in the second carrier matrix supply channel K2. Downstream of the valves W3 and W4, the two carrier matrix supply channels K1 and K2 are combined in a combining and discharge unit VA, AK. This combining and discharge unit VA, AK comprises here a combining section VA in which the two supply channels are combined and also a line section AK (discharge channel) disposed downstream therefrom, via which line section the combined carrier matrix flows are supplied to a subsequently connected usage device (here a testing device for catalytic and/or adsorbtive/absorbent systems and/or a chemical analysis device. In the illustrated case, merely the channel K1 has a metering device D; however one such can also be disposed in addition in the channel K2.

In the illustrated case, how a dilution step is produced in the device according to the invention is now described subsequently. Basically, a metering device which is configured according to the present invention for controlled loading and conducting of the carrier matrix flow, with respect to the flow configuration thereof, comprises at least two matrix flow inlet units (here channel K1 and channel K2). As illustrated in the present case, the carrier matrix source 0 can hereby be the same for both matrix flow channels. K1, K2, i.e. for all inlets, however it is also possible to use different, i.e. separate, carrier matrix sources per channel. In each of the two matrix flow channels K1 and K2, a suitable measuring and control device F is provided, with which the matrix flow (in particular the througflow volume per unit of time) can be detected and controlled. These units F1 and F2 are operated electronically here, however manual devices are also conceivable. In the present case, the device F1 is disposed in the first channel K1 upstream of the metering device D, however it is also conceivable to dispose the device F1 after the metering device D.

By means of the metering device D which is configured in turn as a piezoelectrically activated droplet emission unit, the material to be metered is added to the carrier matrix flow conducted in the channel K1. Shortly thereafter, the concentration measurement of metered material in the carrier matrix flow of the channel K1 is effected by means of the measuring device M. The spacing between droplet emission point or metering device D and measuring device M must be chosen to be so short that as few as possible contamination effects can occur in the connection line but a homogeneous distribution of the material to be metered is still ensured in the carrier matrix.

In order to prevent or to reduce any possibly necessary flow towards the measuring device, according to the configuration of the measuring device M, a shut-off device in the form of a valve W1 is disposed, in the present case, between measuring device and the part of the channel K1 situated downstream of the device D. The measuring device M can be placed, alternatively to the illustrated positioning, also at the point of the actual use of the system (i.e. at the outlet of the discharge channel AK). However, such small material concentrations possibly prevail there that these can no longer be detected by the commercially available measuring technology. Also too high concentrations can then be recognised only at the end of the system and possibly cannot be prevented in a timely manner or are discharged (via the reject channel SK).

As shortly as possible after the measuring unit M or the location of the droplet emission D, a conduction path SK is fitted in order to discharge carrier matrix not provided for use from the first carrier matrix supply channel K1. The spacing between droplet emission point D and outlet SK must hereby be chosen to be as short as possible in order to minimise any possible contamination effects in the connection line.

In order to be able to open and close the waste air line or the reject channel SK according to requirements, the shut-off device W2 is disposed (alternatively or additionally a control device can also be provided).

Furthermore, it is ensured by providing the further valve W3 that a carrier matrix flow loaded in an undesired manner does not reach the combining section VA or the discharge channel AK via the channel K1. The further shut-off and/or control device W3 hence separates the loaded and the unloaded carrier matrix flow (supplied via the carrier matrix supply channel K2). It is also possible to configure the shut-off and/or control devices or valves W2, W3 and W4 for waste air and between loaded and unloaded carrier matrix flow as a single shut-off and/or control device, for example in the form of a three-way valve. Care must hereby be taken that the carrier matrix flow is interrupted briefly during the switching process, which leads to a short-term rise in pressure at the point of the droplet emission. This can disrupt the droplet emission process.

In order to avoid the above-described contamination effects, it is furthermore sensible, in addition to the use of as short as possible conduction paths in the channels K1 and K2 (not shown here), to heat the relevant conduction paths (for example by means of a heating device). However, it can also be adequate here, as an alternative, to keep the temperature of the carrier matrix flow which prevails at the point of the droplet emission D constant in the following lines. This can take place for example by providing a temperature-insulating covering. It must thereby be ensured in particular that the line walls do not fall below the temperature. This can be achieved for example by a sufficiently thick insulating layer.

Through at least one further line, here the second carrier matrix supply channel K2, including the units disposed therein, at least one further unloaded carrier matrix flow is now conducted, according to the invention, to the loaded carrier matrix flow of the channel K1 (cf. also subsequent embodiment 6), which leads to dilution of the loaded carrier matrix flow corresponding to the ratio of the two volume flows of the channels K1 and K2. It is necessary for calculation of the dilution to know the carrier matrix volume flow supplied by means of the channel K2 and to have the possibility of controlling the latter. For this purpose, a suitable measuring and control device F2 for the matrix flow is provided in the channel K2. This can also be operated electronically, manually or in a mixed form, just like the device Fl. In order to be able to interrupt or control the dilution flow if necessary, the shut-off and/or control device W4 is integrated in the line K2, as described. The carrier matrix flows of the two channels, guided together in the combining section VA can, if the desired concentration of substance to be metered is adjusted correctly, then be supplied for the subsequent application via the discharge channel AK.

### Embodiment 6

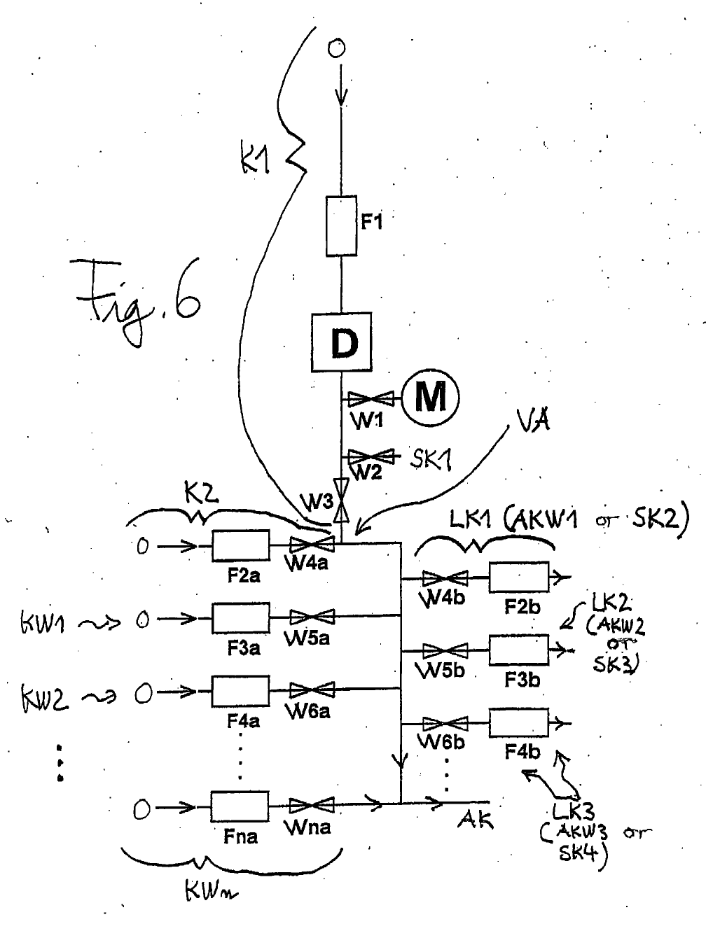

FIG. 6 shows a further embodiment of a device for metering a material into a carrier matrix, which is configured basically just like the embodiment shown in FIG. 5. Therefore only the differences are described subsequently. The basic idea hereby is of extending the system presented in FIG. 5 by further dilution steps: for this purpose, the combining and discharge unit VA, AK has a plurality of inflow channels KWn (n=1, 2, . . . ) which flow in downstream of the combining section VA and upstream of the discharge channel AK. Each of these inflow channels KWn is connected to the carrier matrix source 0 for supplying a carrier matrix flow into the respective inflow channel. Furthermore, each of these inflow channels KWn respectively has a measuring and/or control device Fna (n=3, 4, . . . ) and, downstream of this measuring and/or control device, respectively one shut-off unit (valve) Wna (n=5, 6, . . . ).

The presented system likewise has a plurality of outlet channels LKn (n=1, 2, . . . ) which flow out downstream of the combining section VA and upstream of the discharge channel AK. Via these outlet channels LKn, respectively a part of the already combined carrier matrix flows of the first and of the second carrier matrix supply channel and also of the inflow channels which already flow in possibly in front of the respective outlet channel LK can be discharged. Each of these outlet channels LKn has respectively one shut-off device (valve) Wnb (n=4, 5, . . . ) and also, downstream thereof, respectively one measuring and/or control device Fnb (n=2, 3, 4, . . . ).

The inflowing inflow channels KWn and also the ouffiowing outlet channels LKn are disposed respectively alternately, in the present case there follows, in the downstream direction after the combining section VA, firstly a first outlet channel LK1, then a first inflow channel KW1, then a second outlet channel LK2 etc. According to the requirements of the system, such an outlet channel LKn can be configured either as a further discharge channel AKWn (n=1, 2, . . . ), via which the carrier matrix flows already combined upstream can be supplied at least partially to a subsequently connected use (e.g. chemical analysis device or previously mentioned test device), or be configured as reject channel SKn (n=2, 3, . . . ), via which the carrier matrix flows already combined upstream can be discharged at least partially without being supplied for subsequent use.

The measuring and/or control devices F1, F2a (in the first or second carrier matrix supply channel K1 or K2), F3a, F2b, F4a, F3b, . . . , which are used in the channels of the above-described system, are thereby configured in the present case such that, with them, the carrier matrix volume flows which flow through the respective channel can be detected. The respective measuring results are combined in a central calculating unit, not shown here, so that the concentrations in the then respectively diluted carrier matrix resulting respectively after the inflowing or outflowing channels can be calculated with sufficient accuracy. With reference to the calculated concentrations, the supplied or discharged carrier matrix flows can in turn be controlled via the measuring and/or control devices of the individual channels.

In the illustrated example, the basic system presented with reference to FIG. 5 is hence extended by a plurality of dilution steps: this has the advantage that even the smallest concentrations of metered material can be generated. A separate shut-off and/or control device (W4a, W5a, W6a, . . . ) was thereby integrated for each dilution matrix flow. In order to calculate the dilution, it is necessary, as described, to know the respectively supplied (or discharged) carrier matrix volume flow and to have the possibility of controlling this. For this purpose, the measuring and/or control devices are provided for the respective matrix flow. After each dilution step, a discharge of carrier matrix flow via the channels LKn is possible in the presented example. Also these channels respectively have separate shut-off devices W4b, W5b, . . . . Since the respectively discharged carrier matrix volume flow can be detected via the measuring and control devices F2b, F3b, . . . , it is possible to determine the dilution present downstream of the respective outlet channel LK and to control the individual flows suitably. As already described, the respectively discharged carrier matrix flow can be supplied either for use (configuration of the respective outlet channel LK as further discharge channel AKW) or it can also be discharged without use (configuration of the respective outlet channel LK as reject channel SK).

The principles and application forms described in the above embodiments can be used in many technical spheres. Coordination of the individual components is sensibly effected adapted to the respective purposes of use. The individual components can be operated separately from each other, manually or also via separate control and regulation units. It is also possible to combine the illustrated entire system (for example in FIG. 6) in a single measuring and control loop, by means of which all the measuring and/or control devices F used, all the shut-off and/or control devices W or V and also the metering device D and the concentration measuring unit M can be controlled and regulated. The possibility of automation of the operation and the computer-aided evaluation of the detected crude date is thereby advantageous.

1. Device for metering a material into a carrier matrix, comprising
a carrier matrix source (0) for producing and/or supplying a carrier matrix,
a supply vessel (1) which can be filled and/or is filled with the material in liquid form,
a metering unit (3, 4) comprising a metering chamber (4), which is disposed downstream of the carrier matrix source, and a metering head (3) which is disposed downstream of the supply vessel and configured for piezoelectrically activated droplet emission, the carrier matrix being able to be supplied to the metering chamber from the carrier matrix source and the material being able to be supplied to the metering head from the supply vessel, being able to be emitted via the metering head into the metering chamber and consequently being able to be metered into the carrier matrix,
and comprising
a pressure control device which is configured to control at least one of the pressure in the supply vessel (1) and the pressure in the metering unit.

2. Device according to claim 1,
wherein
the pressure control device is configured to control at least one of the pressure prevailing in the supply vessel in the gas phase above the material to be metered, the pressure prevailing in the metering chamber and the pressure difference between these two pressures.

3. Device according to claim 1,
wherein
the pressure control device has a transfer line (Ü) which connects the supply vessel to the metering chamber in an open gas manner.

4. Device according to claim 3,
wherein
the transfer line has a material shut-off device (5) with which a gas exchange between the metering chamber and the supply vessel is possible and with which passage of the material between the metering chamber and the supply vessel can be prevented.

5. Device according to claim 4,
wherein
the material shut-off device comprises at least one of:
a path section of the transfer line which has a substance which performs at least one of adsorbs the material, absorbs the material and acts catalytically on the material,
a membrane in the transfer line,
and
a path section of the transfer line filled with a liquid

6. Device according to claim 3,
wherein
the transfer line has a shut-off device (V1) with which at least one of a gas exchange and a passage of the material between the metering chamber and the supply vessel can be prevented.

7. Device according to claim 1,
wherein
the pressure control device has at least one pressure sensor (6), which is connected to at least one of the supply vessel and the metering chamber, for detecting the pressure in the supply vessel and/or in the metering chamber, a control unit (7) connected to the pressure sensor (6) and an adjustment unit (8, 10, 11, 12) connected to the control unit, a pressure in the supply vessel and/or a pressure in the metering chamber being able to be adjusted with the control unit as a function of the pressure detected in the supply vessel and/or in the metering chamber via the adjustment unit.

8. Device according to claim 7,
wherein
the pressure control device has a pressure sensor (6b) connected to the supply vessel and a pressure sensor (6a) connected to the metering chamber for detecting the pressures in the supply vessel and in the metering chamber.

9. Device according to claim 7,
wherein
a pressure can be adjusted in the supply vessel by the control unit and via the adjustment unit.

10. Device according to claim 7,
wherein
the adjustment unit has a pressure supply line (11) which can be supplied with at least one of a high pressure and a low pressure and is connected to at least one of the supply vessel and to the metering chamber.

11. Device according to claim 10,
wherein
the pressure supply line has at least one shut-off device (V2, V3) with which the supply of the pressure supply line with a high pressure and/or with a low pressure can be switched on and off.

12. Device according to claim 7,
wherein
the adjustment unit has at least one of a pump (8p) connected to at least one of the supply vessel and the metering chamber, and a piston (8k) connected to at least one of the supply vessel and the metering chamber.

13. Device according to claim 12,
wherein
at least one line (12) which has a shut-off device (V4) between at least one of the pump and piston, and the supply vessel and metering chamber.

14. Device according to claim 7,
wherein
at least one throughflow line (2) is connected to the supply vessel (1) and to the metering head (3) for conducting material to be metered from the supply vessel to the metering head, the adjustment unit having at least one liquid pump (10) which is disposed on or integrated in this throughflow line, which liquid pump is configured to pump material to be metered from the supply vessel to the metering head and in the reverse direction.

15. Device according to claim 14,
wherein the at least one liquid pump comprises
a first and a second liquid pump, the first pump being configured to pump material to be metered from the supply vessel to the metering head and the second pump being configured to pump material to be metered in the reverse direction.

16. Device according to claim 14,
further comprising
at least one excess pressure valve (V7) which is disposed at least at one location selected from between the at least one liquid pump and the metering head, between the supply vessel and the at least one liquid pump in or on the throughflow line.

17. Device according to claim 1,
further comprising
a throughflow line (2) connected to the supply vessel (1) and the metering head (3) for conducting material to be metered from the supply vessel to the metering head.

18. Device according to claim 17,
wherein
the throughflow line (2) has at least one shut-off device (V5, V6) for interrupting the material transport between supply vessel (1) and metering head (3).

19. Device according to claim 17,
wherein
at least one filter device is integrated in the throughflow line (2).

20. Device according to claim 17,
further comprising
a pressure control device which is configured to control the pressure resulting from a difference in level between the liquid level of the material to be metered in the supply vessel and the level of the outlet location of the material to be metered in the metering space, in particular a pressure control device which is configured to keep this level difference and/or this pressure constant.

21. Device according to claim 20,
wherein
the pressure control device has a mechanical adjustment device which is configured to keep the difference in level constant.

22. Device according to claim 1,
wherein
the pressure control device has a pump, which is disposed in a throughflow line (2) connecting the supply vessel (1) to the metering head (3), the pump being configured such that, by means of it, the material to be metered can be supplied at the outlet location in the metering head with a constant pressure.

23. Device according to claim 1,
wherein
a lower end of the supply vessel interior is disposed at a greater height than the metering head and the pressure control device has a pressure reducing unit which is disposed in a throughflow line (2) connecting the supply vessel (1) to the metering head (3), which pressure reducing unit is configured such that the pressure resulting due to the difference in height on the material to be metered at the outlet location thereof in the metering head can be reduced to a constant pressure which is less than this resulting pressure.

24. Device according to claim 23,
wherein
the pressure reducing unit has at least one of an excess pressure valve and a needle valve.

25. Device according to claim 1,
wherein
a plurality of supply vessels which are connected to the metering head for containing a material or material mixture in liquid form, the material or material mixtures being able to be supplied to the metering head from the supply vessels, being able to be emitted via the metering head into the metering chamber and being able to be metered into the carrier matrix.

26. Device for metering a material into a carrier matrix, comprising
a carrier matrix source (0) of a carrier matrix,
a first carrier matrix supply channel (K1) to which a carrier matrix can be supplied from the carrier matrix source and which has a first control device (F1) for controlling the carrier matrix flow through the first carrier matrix supply channel and also a metering device (D) which is configured to meter the material into the carrier matrix supplied to the first carrier matrix supply channel,
a second carrier matrix supply channel (K2) to which a carrier matrix can be supplied from the carrier matrix source and which has a second control device (F2) for controlling the carrier matrix flow through the second carrier matrix supply channel, and
a combining and discharge unit (VA, AK) which is disposed at the downstream end of the first and of the second carrier matrix supply channel and has a combining section (VA) in which the carrier matrix flow, which has been supplied with material, of the first carrier matrix supply channel and the carrier matrix flow of the second carrier matrix supply channel can be combined, and a discharge channel (AK) disposed downstream of the combining section via which discharge channel the combined carrier material flows can be supplied at least partially for use.

27. Device according to claim 26,
wherein
the combining and discharge unit has at least one inflow channel (KW) which flows in between the combining section (VA) and the discharge channel (AK) and via which a further carrier matrix can be supplied from the carrier matrix source to the combined carrier matrix flows of the first and of the second carrier matrix supply channel.

28. Device according to claim 27,
wherein
at least one of the inflow channels (KW) has at least one of a measuring device and a control device for measuring and/or controlling the carrier matrix flow through this inflow channel.

29. Device according to claim 26,
wherein
the combining and discharge unit has at least one outlet channel (LK) which flows out between the combining section (VA) and the discharge channel (AK) and via which a part of the combined carrier matrix flows of the first and of the second carrier matrix supply channel can be discharged.

30. Device according to claim 29,
wherein
at least one of the outlet channels (LK) has at least one of a measuring device and a control device for measuring and/or controlling the carrier matrix flow through this outlet channel.

31. Device according to claim 29,
wherein
at least one of the outlet channels (LK) is configured as a further discharge channel (AKW), via which the combined carrier matrix flows can be supplied at least partially for use,
or
as a reject channel (SK) via which the combined carrier matrix flows can be discharged at least partially and without being supplied for use.

32. Device according to claim 29,
further comprising
a plurality of inflow channels (KW) and a plurality of outlet channels (LK), viewed in the flow direction, the inflowing inflow channels and outflowing outlet channels being disposed alternately.

33. Device according to claim 26,
further comprising
a reject channel (SK) which is disposed flowing out in the first carrier matrix supply channel (K1) downstream of the metering device (D) and via which reject channel the carrier matrix flow of the first carrier matrix supply channel can be discharged at least partially and without being supplied for use.

34. Device according to claim 26,
wherein
there is disposed in the flow path of the carrier matrix, at least one of a shut-off and a control unit (W) which is configured to shut off and open or to control the throughflow volume of the carrier matrix flow per unit of time

35. Device according to claim 34,
wherein
at least one of the shut-off or control units (W) is disposed in at least one of the first carrier matrix supply channel (K1), directly upstream of the combining section (VA) in the first carrier matrix supply channel, in the second carrier matrix supply channel (K2), directly upstream of the combining section (VA) in the second carrier matrix supply channel, in the discharge channel (AK), in an outlet channel (LK), in a reject channel (SK), in a further discharge channel (AKW) or in an inflow channel (KW).

36. Device according to claim 26,
wherein
the carrier matrix source has at least one multiple supply unit with which a carrier matrix can be supplied to a plurality of the channels from at least one of the first carrier matrix supply channel (K1), the second carrier matrix supply channel (K2) and the inflow channels (KW)

37. Device according to claim 26,
wherein
the first measuring and control device (F1) is disposed upstream of the metering device (D).

38. Device according to claim 26,
wherein
at least one of the first carrier matrix supply channel (K1) and the discharge channel (AK) has a concentration measuring unit (M) which is disposed downstream of the metering device (D) and configured to measure the concentration of metered material in the carrier matrix flow.

39. Device according to claim 38,
wherein
the concentration measuring unit (M) can be switched by means of a valve, to the first carrier matrix supply channel (K1) or to the discharge channel (AK).

40. Device according to claim 38,
wherein
the concentration measuring unit (M) is configured to measure the concentration without removing a sample of carrier matrix loaded with material
or
by removing a sample of carrier matrix loaded with material.

41. Device according to claim 26,
wherein
at least one of the channels has a temperature control device configured to control the temperature of the throughflowing carrier matrix flow,
or
is provided at least partially with a temperature-insulating covering.

42. Device according to claim 26,
further comprising
a usage device, disposed downstream of the discharge channel (AK) including at least one of a chemical analysis device, a test device and a production device to which the carrier matrix flow discharged from the discharge channel can be supplied for use.

43. Device for metering a material into a carrier matrix according to claim 26,
further comprising
the inclusion of a metering device for metering a material into a carrier matrix, comprising
a supply vessel (1) for supplying the material in liquid form,
a metering unit (3, 4) comprising a metering chamber (4), which is disposed downstream of the carrier matrix source, and a metering head (3) which is disposed downstream of the supply vessel and configured for piezoelectrically activated droplet emission, the carrier matrix being able to be supplied to the metering chamber from the carrier matrix source and the material being able to be supplied to the metering head from the supply vessel, being able to be emitted via the metering head into the metering chamber and consequently being able to be metered into the carrier matrix, and
a pressure control device which is configured to control at least one of the pressure in the supply vessel (1) and the pressure in the metering unit.

44. Device according to claim 43,
wherein
the metering device (D) comprises the supply vessel (1), the metering unit (3, 4) and the pressure control device.

45. Method for metering a material into a carrier matrix, comprising the steps of
filling a supply vessel (1) with the material in liquid form,
conducting a carrier matrix from a carrier matrix source (0) to a metering chamber (4) of a metering unit (3, 4) which is disposed downstream of the carrier matrix source,
conducting the material from the supply vessel to a metering head (3) of the metering unit (3, 4) which is disposed downstream of the supply vessel and
emitting the material via the metering head and of metering the emitted material into the carrier matrix in the metering chamber,
wherein
the pressure in the supply vessel and the pressure in the metering unit is controlled.

46. Method according to claim 45,
wherein
the metering takes place with a metering device comprising
a supply vessel (1) for supplying the material in liquid form,
a metering unit (3, 4) comprising a metering chamber (4), which is disposed downstream of the carrier matrix source, and a metering head (3) which is disposed downstream of the supply vessel and configured for piezoelectrically activated droplet emission, the carrier matrix being able to be supplied to the metering chamber from the carrier matrix source and the material being able to be supplied to the metering head from the supply vessel, being able to be emitted via the metering head into the metering chamber and consequently being able to be metered into the carrier matrix, and
a pressure control device which is configured to control at least one of the pressure in the supply vessel (1) and the pressure in the metering unit, and
the pressure control device is configured to control at least one of the pressure prevailing in the supply vessel in the gas phase above the material to be metered, the pressure prevailing in the metering chamber and the pressure difference between these two pressures.

47. Method for metering a material into a carrier matrix, comprising the steps of
supplying a carrier matrix from a carrier matrix source (0) to a first carrier matrix supply channel (K1), controlling the carrier matrix flow through the first carrier matrix supply channel and metering the material into the carrier matrix supplied to the first carrier matrix supply channel,
supplying a carrier matrix from the carrier matrix (0) to a second carrier matrix supply channel (K1) and controlling the carrier matrix flow through the second carrier matrix supply channel,
combining the controlled material-loaded carrier matrix flow of the first carrier matrix supply channel and the controlled carrier matrix flow of the second carrier matrix supply channel, and
discharging the at least partial supply of the combined carrier matrix flows for use.

48. Method according to claim 47,
wherein
the metering takes place with a device comprising a combining and discharge unit (VA, AK) which is disposed at the downstream end of the first and of the second carrier matrix supply channel and has a combining section (VA) in which the carrier matrix flow, which has been supplied with material, of the first carrier matrix supply channel and the carrier matrix flow of the second carrier matrix supply channel can be combined, and a discharge channel (AK) disposed downstream of the combining section via which discharge channel the combined carrier material flows can be supplied at least partially for use, and wherein
the combining and discharge unit has at least one inflow channel (KW) which flows in between the combining section (VA) and the discharge channel (AK) and via which a further carrier matrix can be supplied from the carrier matrix source to the combined carrier matrix flows of the first and of the second carrier matrix supply channel.

49. (canceled)
